(19) United States
(12) Patent Application Publication
(10) Pub. No.: US 2025/0352585 A1
(43) Pub. Date: Nov. 20, 2025

(54) CHIMERIC OVARIAN FOLLICLE-BASED THERAPY TO TREAT FEMALE INFERTILITY

(71) Applicant: National University of Singapore, Singapore (SG)

(72) Inventors: Rong LI, Singapore (SG); Haiyang WANG, Singapore (SG)

(73) Assignee: National University of Singapore, Singapore (SG)

(21) Appl. No.: 18/866,210

(22) PCT Filed: May 17, 2023
(86) PCT No.: PCT/SG2023/050339

§ 371 Date: Nov. 15, 2024

(30) Foreign Application Priority Data
May 17, 2022 (SG) 10202205146T

(51) Int. Cl. A61K 35/54; C12N 5/075

(52) U.S. Cl. CPC A61K 35/54; C12N 5/0609; C12N 2502/243

(57) ABSTRACT

The present invention relates to methods of rejuvenating aged oocytes using young somatic cells through the generation of chimeric follicles. The chimeric follicles defined herein may be used to treat infertility or improve fertility in female subjects.

### CROSS-REFERENCE TO RELATED APPLICATIONS

This application claims the benefit of priority of Singapore Application No. 10202205146T, filed 17 May 2022, the contents of it being hereby incorporated by reference in its entirety for all purposes.

### FIELD OF THE INVENTION

The present invention is in the field of cell biology. In particular, the present invention relates to methods of rejuvenating aged oocytes. The present invention further discloses the methods of treating infertility or improving fertility using young somatic cells to create a reconstituted chimeric follicle that comprises an aged oocyte and young somatic cells.

### BACKGROUND OF THE INVENTION

The mammalian ovary comprises numerous ovarian follicles which shelter and nourish the oocyte during the development. The mammalian ovarian follicle comprises the oocyte and its surrounding somatic or non-reproductive cells including granulosa cells (GCs) and theca cells. These somatic cells are the smallest functional units to maintain reproductive competency in women which grow and develop with the oocyte. Importantly, the somatic cells especially the GCs are capable of nourishing the developing oocyte through specialized structures known as transzonal projections (TZPs), and providing energy, nutrients, and microenvironment for the development and growth of the oocyte. These allow the oocyte to undergo maturation to be competent for fertilization, resulting in a viable embryo. Additionally, the GCs produce the female hormone estrogen which is vital for reproductive health in women.

A recent report has stated that infertility affects 1 in 6 people globally and age-related decline female fertility is a critical global social and clinical issue. Maternal aging has deleterious effects on fertility either spontaneously (naturally) or via assisted reproductive techniques (ART)/in vitro fertilization (IVF), due to a gradual loss in the number and quality of ovarian follicles with age and the limitations of ART. Maternal aging is associated with senescence of apoptosis of somatic cells, resulting in atresia of the ovarian follicles.

To improve fertility in female subjects, the existing method to rejuvenate aged oocytes is mitochondrial transfer which works by the replacement of the damaged mitochondria in an aged oocyte from a recipient female subject with healthy mitochondria from a donor female subject. This method is controversial and raises safety concerns as donor mitochondrial DNA is transferred to the developing embryo.

Therefore, there is a need to develop new methods of rejuvenating aged oocytes to prevent and treat infertility that improves the number and competence of oocytes without transferring the DNA of the donor to the oocyte of the recipient.

### SUMMARY

In one aspect, there is provided an in vitro method of rejuvenating an aged oocyte comprising combining the aged oocyte with a donor ovarian follicle that does not comprise an oocyte to generate a chimeric ovarian follicle.

In another aspect, there is provided a chimeric ovarian follicle, comprising an aged oocyte and donor somatic cells.

In another aspect, there is provided a method of treating infertility or improving fertility in a female subject in need thereof, comprising:
a) obtaining an ovarian follicle from the subject and isolating an aged oocyte from an ovarian follicle:
b) obtaining an ovarian follicle from a donor and removing an oocyte from the donor ovarian follicle:
c) transplanting the aged oocyte from a) with the ovarian follicle without the oocyte from b) to generate a chimeric ovarian follicle:
d) culturing the chimeric ovarian follicle to generate a mature chimeric ovarian follicle; and
e) i) implanting the chimeric ovarian follicle into the female subject to treat infertility or improve fertility: or
ii) ovulating a mature oocyte from the mature chimeric ovarian follicle and isolating the mature oocyte for in vitro fertilization.

### Definitions

As used herein is the term “rejuvenating” in the context of an oocyte refers to the process of restoring the microenvironment of an aged oocyte to promote development, growth and maturation of the oocyte, such that the oocyte has potential to develop into a viable embryo.

The term “folliculogenesis” refers to development and maturation of the ovarian follicle from the primordial stage to the primary stage, the secondary stage, the early antral (pre-antral) stage and the antral stage. The ovarian follicle comprises an oocyte and somatic cells. These somatic cells may comprise but are not limited to cumulus cells, granulosa cells and theca cells. The ovarian follicle at each stage may be characterized by the presence of one or more specific cell types and/or phenotypes. For example, an ovarian follicle at the primordial stage may be characterized by the presence of an oocyte surrounded by pre-granulosa cells, an ovarian follicle at the primary stage may comprise an oocyte surrounded by a single layer of granulosa cells and an ovarian follicle at the secondary stage may comprise an oocyte surrounded by more than one layer of granulosa cells. The ovarian follicle at the secondary stages matures into the pre-antral stage which comprises an oocyte, more than one layer of granulosa cells and theca cells. The pre-antral stage ovarian follicle subsequently progresses to the antral stage which is characterized by the formation of an antrum within the somatic cells comprising more than one layer of granulosa cells and theca cells, surrounding an immature oocyte. The ovarian follicle at the antral stage refers to a pre-ovulatory follicle or a mature follicle, and the immature oocyte in the pre-ovulatory follicle may be a fully grown germinal vesicle (GV) stage oocyte.

As used herein, the term “chimeric ovarian follicle” refers to a follicle comprising an oocyte obtained from of an ovarian follicle of a first subject and somatic cells obtained from an ovarian follicle of a second subject. The first subject may also be known as a “recipient” and the second subject may also be known as a “donor”. The terms “chimeric ovarian follicle”.

“chimeric follicle”, “reconstituted chimeric ovarian follicle” and “reconstituted chimeric follicle” can be used interchangeably in this context.

The term “somatic cells” refers to non-reproductive cells. In the context of this invention, “somatic cells” refer to the non-reproductive somatic cells within an ovarian follicle that surround an oocyte during folliculogenesis. These cells may comprise but are not limited to pre-granulosa cells, cumulus cell, granulosa cells and theca cells. For example, at the primordial stage of folliculogenesis, somatic cells comprise pre-granulosa cells, at the primary and secondary stages, somatic cells comprise granulosa cells, and at the early antral (pre-antral) and antral stages, somatic cells comprise granulosa cells and theca cells. These cells surrounding the oocyte divide undergo cell division during the development of the oocyte.

As used herein, the term “aged” with respect to an ovarian follicle refers to an ovarian follicle that has deteriorated in quality. One measure of the quality of an ovarian follicle is the ability of the ovarian follicle to develop and mature during folliculogenesis. As such, an aged ovarian follicle is an ovarian follicle that is incapable or less capable of developing into a mature follicle to induce ovulation of an oocyte. An aged ovarian follicle comprises an aged oocyte and somatic cells that are less or not capable of promoting development of an oocyte.

As used herein, the term “young” with respect to an ovarian follicle refers to an ovarian follicle that is capable of developing into a mature follicle during folliculogenesis.

“Mature ovarian follicle” refers to an ovarian follicle at the antral stage of follicle development which comprises granulosa cells and a fully grown germinal vesicle stage oocyte. The terms “mature ovarian follicle” and “preovulatory ovarian follicle” can be used interchangeably in this context.

“Fully grown germinal vesicle stage oocyte” refers to an oocyte that is immature and has the ability to undergo maturation.

“Mature oocyte” refers to a metaphase II (MII) arrested oocyte or egg that has undergone meiotic maturation and has the ability to undergo fertilization. The terms “mature oocyte”, “ovulated oocyte” and “MII oocyte” can be used interchangeably in this context.

As used herein, the term “competence” refers to the ability of an oocyte to develop, grow and mature for fertilization which results in a viable embryo. A viable embryo may progress and develop into a fetus in the womb.

The term “subject” refers to a human or non-human mammal. Examples of such mammals include but are not limited to a primate, a mouse, a rat, a guinea pig, a rabbit, and a dog.

The term “infertility” refers to a disease of the female reproductive system that is characterized by inability or reduced ability of the oocyte to be fertilized by male reproductive cells or the inability or reduced ability of the fertilized oocyte to grow and develop into a viable embryo.

As used herein, the term “fertile” refers to the ability of the oocyte to be fertilized by male reproductive cells or the ability of the fertilized oocyte to grow and develop into a viable embryo.

As used herein, the term “about”, in the context of concentrations of components and percentages of compounds, typically refers to +/−5% of the stated value, +/−4% of the stated value, more typically +/−3% of the stated value, more typically, +/−2% of the stated value, even more typically +/−1% of the stated value, and even more typically +/−0.5% of the stated value. Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

### BRIEF DESCRIPTION OF THE DRAWINGS

The invention will be better understood with reference to the detailed description when considered in conjunction with the non-limiting examples and the accompanying drawings, in which:

FIG. 1 shows the development of ovarian follicle, the maturation of oocyte during folliculogenesis, and the interaction of the oocyte with somatic cells within the ovarian follicle. FIG. 1A shows an overview of follicle development, oocyte maturation and early embryo development. FIG. 1B shows the physical interaction between the oocyte and granulosa cells within the ovarian follicle during folliculogenesis. The follicle comprises somatic or non-reproductive granulosa cells which nourish the oocyte through specialized structures known as Transzonal Projections (TZPs).

FIG. 2 shows the size and quality of young and aged ovarian follicle in a three-dimensional (3D) culture. FIG. 2A shows that aged follicles grow slower as compared to young follicles and FIG. 2B shows that the size of the aged follicles is decreased as compared to the size of the young follicles. FIG. 2C shows that aged follicles are more prone to undergo atresia during in vitro culture when compared to young follicles. Young follicles maintained their 3D structure with proliferation of GCs and antrum formation (white arrow) while being cultured and encapsulated in hydrogels of alginate-reconstituted basement membrane (rBM) interpenetrating network (Alg-rBM IPN). In contrast, aged follicles experience increased atresia during in vitro culture (right panel of FIG. 2C). Follicles are considered atretic if there was disruption of contact between the oocyte (white arrowhead) and granulosa cells, leading to the release of oocytes from the follicles (top right panel), or if the follicles contained apoptotic or dead oocytes (bottom right panel). FIG. 2D shows that the death rate of the aged follicles is significantly higher as compared to the young follicles.

FIG. 3 shows the generation of chimeric follicles to rejuvenate aged oocytes. FIG. 3A shows the method to generate chimeric follicles through the implantation of an aged oocyte into a young follicle. FIG. 3B shows that the generated chimeric follicles develop and grow to the size of 300 μm in diameter and FIG. 3C shows that the generated chimeric follicles progress in development and develop to antral stage chimeric follicles, evidenced by the formation of an antrum. FIG. 3D shows the maturation rate of oocytes in a chimeric follicle comprising a young oocyte and young GCs (YY).

FIG. 4 shows that the chimeric follicles generated through the transplantation of aged oocytes in young follicles improve the quality of these aged oocytes. FIG. 4A shows the three criteria used to determine the quality of the aged oocytes which are the morphology of the spindles, the segregation of the chromosomes and the distribution of mitochondria in the oocyte. FIG. 4B shows that culturing the aged oocytes in young follicles within the chimeric follicle (AY) significantly restored meiotic maturation of aged oocytes to a level similar to young oocytes grown in young follicles within the chimeric follicle (YY). FIG. 4C shows representative live cell images of meiotic spindle and chromosomes in mature oocytes from chimeric follicles comprising aged oocytes and young somatic cells (AY) and chimeric follicles comprising aged oocytes and aged somatic cells (AA). The left panel of FIG. 4C shows an example of chromosomal misalignment in aged oocyte from the AA reconstituted chimeric follicle and the image in middle panel of FIG. 4C shows an example of abnormal spindle in an aged oocyte from the AA reconstituted chimeric follicle, while the image in right panel of FIG. 4C shows example of a normal spindle morphology (white arrow) and chromosomes alignment (white arrowhead) in aged oocyte from AY reconstituted chimeric follicle. FIG. 4D shows the quantification of the percentage of chromosomal misalignment on the metaphase plate in AA reconstituted chimeric follicles and AY reconstituted chimeric follicles. FIG. 4E shows the spindle abnormalities in oocytes from AA reconstituted chimeric follicles and AY reconstituted chimeric follicles. Disorganized spindle and misaligned chromosomes were present in aged oocytes from AA reconstituted chimeric follicles, but the occurrence of aberrant spindle and chromosome alignment was significantly reduced in oocytes from AY reconstituted chimeric follicles. FIG. 4F shows the confocal microscopy images of mitochondrial distribution patterns in oocytes from reconstituted chimeric follicles comprising 1) young oocyte and young granulosa cells (YY), 2) aged oocyte and aged granulosa cells (AA) and 3) aged oocyte and young granulosa cells (AY). Mitochondria were detected by using MitoTracker Red CMXRos. FIG. 4G shows that the average size of mitochondria cluster in oocytes from 1) YY reconstituted chimeric follicles, 2) AA reconstituted chimeric follicles and 3) AY reconstituted chimeric follicles. The size of mitochondria cluster in oocytes from AY reconstituted chimeric follicles is significantly reduced as compared to the oocytes from AA reconstituted chimeric follicles.

FIG. 5 shows the effect of young GCs on aneuploidy in age oocytes. FIG. 5A shows the 3D reconstruction of chromosomes and kinetochores in oocytes from AA reconstituted chimeric follicles, and AY reconstituted chimeric follicles. FIG. 5B shows a significant decrease in the percentage of chromosomal abnormalities in the chimeric follicles as compared to the aged follicles.

FIG. 6 shows the effect of young GCs on embryonic development. FIG. 6A shows the schematic diagram of the process of rejuvenating the aged oocyte with young GCs to embryonic development of fertilized eggs. FIG. 6B shows a higher percentage of blastocyst 20) formation in aged oocytes grown with young GCs (AY) as compared to aged oocytes grown with aged GCs (AA). FIG. 6C shows the immunofluorescence images of the blastocyst resulting from 1) YY reconstituted chimeric follicles, 2) AA reconstituted chimeric follicles and 3) AY reconstituted chimeric follicles. FIG. 6D shows that the size and total cell number of blastocysts developed from AY chimeric follicles are significantly improved as compared to those from AA chimeric follicles. FIG. 6E shows that oocytes from AY reconstituted chimeric follicles produces a significantly higher number of live pups as compared to AA reconstituted chimeric follicles. The representative images of surrogate mother with the transplantation of the AY and AA reconstituted chimeric follicle and the pups from the AY and AA reconstituted chimeric follicles are shown in the left panel of FIG. 6E. The two pups are delivered after transplanting 20 2-cell stage embryos derived from AY reconstituted chimeric follicles into a surrogate mother, whereas no pup is delivered after transplanting 20 AA 2-cell stage embryos derived from AA chimeric follicles. Middle panel of FIG. 6E shows live birth rates of pups after transferring 2-cell embryos from YY, AA, and AY oocyte into surrogate mothers. Right panel of FIG. 6E shows day I and day 16 pups generated from oocytes obtained from YY and AY chimeric follicles

FIG. 7 shows the possible mitochondrial transport from granulosa cells to oocyte in the chimeric follicles using MTS-mCherry-GFP₁₋₁₀ transgenic mice. FIG. 7A shows the experimental design to study mitochondrial transport within the chimeric follicles which are created using somatic cells from transgenic MTS-mCherry-GFP₁₋₁₀ mice expressing mitochondria-targeted mCherry, and unlabelled oocytes from wild-type mice. FIG. 7B shows the absence of mitochondria transport from the surrounding somatic cells to the oocyte in the chimeric follicles.

### DETAILED DESCRIPTION OF THE PRESENT INVENTION

The present invention refers to a follicular somatic cell-based therapy for treating female infertility.

In a first aspect, the present invention refers to an in vitro method of rejuvenating an aged oocyte comprising combining the aged oocyte with a donor ovarian follicle that does not comprise an oocyte to generate a chimeric ovarian follicle.

In one example, the aged oocyte is obtained from an ovarian follicle of a subject. The ovarian follicle may be an aged ovarian follicle which has deteriorated in quality. Quality of an ovarian follicle may be determined by one or more characteristics or phenotypes. Examples of characteristics or phenotypes that may be used to determine the quality or age of an ovarian follicle include but are not limited to the formation of transzonal projections (TZPs), the ability of somatic cells to promote development of the oocyte, the progression of the ovarian follicle to the antral stage and the number of mature oocytes produced per ovulation cycle.

In one example, an aged ovarian follicle may have poorly formed TZPs, delayed or failure of the ovarian follicle to develop to a mature ovarian follicle or to the antral stage, slower growth rate, decreased number of TZPs and number of mature oocytes produced per ovulation cycle and increased apoptotic or atretic rates relative to a young ovarian follicle, comprise somatic cells that may not be undergoing active division or may be undergoing less active division, and may have increased apoptosis relative to the somatic cells of a young ovarian follicle, or combinations thereof. The aged oocyte of the invention may be obtained from an ovarian follicle of a subject suffering from infertility or reduced fertility.

In some examples, the subject may be suffering from infertility or reduced fertility due to advanced age. In other example, the subject may be suffering from infertility or reduced fertility due to reasons other than advanced age, such as obesity and/or conditions including but not limited to premature ovarian failure, polycystic ovarian syndrome, endometriosis and uterine fibroids. It is generally understood that in the context of fertility advanced age refers to an increased chronological age. In one example, the subject may have a chronological age of about or more than 35 years old. For example, the female subject may be about 35 years old, 40 years old, 45 years old, 50 years old, 55 years old, 60 years old, 65 years old or 70 years old.

It will be understood that the causes of infertility are not exhaustive and may comprise any reason that results in the inability of the oocyte to be fertilized by the male reproductive cells or the fertilized oocyte to grow and develop into a viable embryo.

The aged oocyte may be an oocyte that has undergone deterioration in quality. One measure of the quality of an oocyte is the ability of the oocyte to develop and mature during folliculogenesis. As such, an aged oocyte refers to an oocyte that is incompetent or less competent to develop and mature during folliculogenesis. Phenotypes that can be used to determine the competence of the oocyte to develop into a mature oocyte may comprise but are not limited to morphology of the spindles, alignment of the chromosomes, distribution of mitochondria, chromosome cohesion and level of ATP. The aged oocyte may display spindle abnormalities, chromosomes misalignment, abnormal mitochondria distribution, altered chromosome cohesion and altered level of ATP. It is well known in the art that spindle abnormalities comprise asymmetrically shaped spindles with more than two spindle poles, chromosomes are not aligned in the centre of the oocyte, mitochondria is aggregated in the cytoplasmic compartment, chromosome cohesion is absent or decreased relative to young oocyte and the level of ATP is decreased relative to the young oocyte.

The aged ovarian follicle from which the aged oocyte is obtained may comprise somatic cells that are less capable or not capable of promoting development of the oocyte. The inability or reduced ability of the somatic cells to promote development of the oocyte may be due to deterioration in quality of the somatic cells. The ability of the somatic cells to promote the development of the oocyte may be determined by one or more of reactive oxidative stress level, mitochondria membrane potential, DNA damage and apoptosis.

In one example, the somatic cells that are less capable or not capable of promoting the development of the oocyte are characterized by one or more characteristics or phenotypes selected from the group consisting of higher reactive oxidative stress level, lower mitochondria membrane potential, increased DNA damage and apoptosis, decreased ATP levels, absence of or decreased cohesion, or a combination thereof, relative to somatic cells that are capable of promoting development of the oocyte.

In the method of the present invention, the aged oocyte obtained from the aged ovarian follicle is combined with a donor ovarian follicle that does not comprise an oocyte.

In one example, the donor ovarian follicle may be a young follicle that is obtained from a fertile subject. The fertile subject may have a chronological age of about or less than 35 years old. The subject may be about 35 years old, 30 years old, 25 years old or 20 years old. In another example, the subject is not suffering from diseases or conditions associated with infertility.

The donor ovarian follicle may be identified by one or more characteristic or phenotypes comprising but not limited to well formed transzonal projections (TZPs), somatic cells capable of promoting development of an oocyte that are actively dividing and have no death rate or low death rate as compared to an aged ovarian follicle, and the young ovarian follicle may progress to the antral stage during folliculogenesis or progress to the antral stage within an expected period of time.

The somatic cells of the donor ovarian follicle that are capable of promoting development of the oocyte have one or more predetermined phenotypes selected from the group consisting of lower reactive oxidative stress level, higher mitochondria membrane potential, lesser DNA damage and apoptosis, increased ATP levels, increased cohesion, or a combination thereof, relative to somatic cells that are less capable or not capable of promoting development of the oocyte.

In one example, the aged ovarian follicle from which the aged oocyte is obtained is selected from the group consisting of a primordial follicle, a secondary follicle, an early antral follicle and an antral follicle. It would generally be understood that the primordial follicle is an ovarian follicle at the primordial stage of folliculogenesis, the secondary follicle is an ovarian follicle at the secondary stage of folliculogenesis, the early antral follicle is an ovarian follicle at the early antral stage of folliculogenesis and the antral follicle is at the antral stage of folliculogenesis.

The ovarian follicles of the present invention may be obtained from a subject by means that are known in the art. In one example, the ovarian follicles may be obtained by enzymatic digestion or manual dissection or both. The ovarian follicles may be obtained from the ovaries by manual dissection of the ovaries obtained from the subject using a needle and incubation of the ovaries in a medium comprising an enzyme to isolate the ovarian follicle. The enzyme may be collagenase. In another example, the ovarian follicle may be obtained through manual dissection through defolliculation of ovaries obtained from the subject using a needle. Similarly, an oocyte may be obtained or removed from an ovarian follicle obtained from a subject by means that are known in the art. The oocyte may be obtained or removed from the ovarian follicle manually or through enzymatic digestion. In one example, the oocyte may be obtained or removed from the ovarian follicle by incubating of the oocyte in a media comprising an enzyme that is capable of digesting the cells surrounding the oocyte. The enzyme may be trypsin or collagenase. In another example, the oocyte may be obtained or removed from the ovarian follicle by manual means comprising mouth pipetting. In one example, the subject is a female subject.

The aged oocyte obtained from an aged ovarian follicle is combined with a donor ovarian follicle that does not comprise an oocyte to generate a chimeric ovarian follicle. The aged oocyte obtained from the aged ovarian follicle is manually combined with the donor ovarian follicle that does not comprise an oocyte to generate a chimeric ovarian follicle. In one example, the aged oocyte is transplanted into the ovarian follicle that does not comprise an oocyte using a mouth pipette by picking up the oocyte and releasing the oocyte into the ovarian follicle to generate the chimeric follicle.

In one example, the follicles isolated from the subject and the donor, and the chimeric ovarian follicle may be cultured as a three-dimensional culture. In one example, the chimeric ovarian follicle is cultured in alginate gel or Alg-rBM IPN. In one example, the Alg-rBM IPN contains alginate and basement membrane proteins. In one example, the concentration of the alginate gel is between about 0.1% to 0.5%. The concentration of the alginate gel may be about 0.1%, about 0.2%, about 0.3%, about 0.4% or about 0.5%. In one preferred example, the concentration of the alginate gel is about 0.3%.

In one example, the ovarian follicle from the subject and the donor ovarian follicle are cultured in a first culture medium and the chimeric ovarian follicle is cultured in a second culture medium. The culture media may comprise but is not limited to α-Minimum Essential Medium (αMEM), follicle-stimulating hormone (FSH), bovine serum albumin (BSA), fetal bovine serum (FBS), ITS, fetuin and an oocyte derived growth factor, and combinations thereof. In one example, the first culture medium comprises αMEM, FSH, BSA, ITS and fetuin. In one example, the second culture medium comprises αMEM, FSH, BSA, ITS, fetuin and growth differentiation factor 9 (GDF9). In another example, the chimeric follicle may be cultured in a culture medium comprising αMEM, FSH, BSA and ITS. In another example, the ovarian follicle may be cultured in a culture medium comprising αMEM, FSH, BSA and ITS.

In one example, the chimeric ovarian follicle may be cultured in the second culture medium to generate a mature chimeric ovarian follicle and may be induced to ovulate the mature oocyte from the mature chimeric ovarian follicle. In one example, the mature chimeric ovarian follicle comprises a fully grown germinal vesicle (GV) stage oocyte and somatic cells. The fully grown germinal vesicle stage oocyte would be understood to be an immature oocyte. It will generally be understood that an immature oocyte is incapable of being fertilized and would need to undergo meiotic maturation to form a mature oocyte. In one example, the mature oocyte may be induced with human chorionic gonadotropin (hCG) for ovulation. The ovulated mature oocyte may then be fertilized by male reproductive cells and/or may be utilized in assisted reproductive technologies comprising in vitro fertilization and intracytoplasmic sperm injection.

In one example, the mature chimeric ovarian follicle is an antral stage chimeric follicle.

In one example, the somatic cells comprise granulosa cells.

The quality of the mature chimeric ovarian follicle may be determined using one or more predetermined phenotypes. The one or more predetermined phenotypes may comprise but not limited to the presence of a fully grown GV oocyte, formation of the antrum and combinations thereof. In one example, the mature chimeric ovarian follicle may be characterized by presence of a fully grown GV oocyte and formation of the antrum.

In one example, the aged oocyte is a mammalian oocyte and the donor ovarian follicle is a mammalian ovarian follicle. In one preferred example, the mammalian oocyte is a human or rodent oocyte. In another preferred example, the mammalian donor ovarian follicle is a human or rodent ovarian follicle.

In another aspect, the present invention refers to a chimeric ovarian follicle, comprising an aged oocyte and donor somatic cells. Without being bound by theory, the donor somatic cells in the chimeric ovarian follicle rejuvenate the aged oocyte such that the aged oocyte is able to develop and mature.

In one example, the aged oocyte may be obtained from an aged ovarian follicle comprising somatic cells that are less capable or incapable of promoting development of the oocyte. In one example, the donor somatic cells are obtained from a young ovarian follicle comprising somatic cells that are capable of promoting development of the oocyte, wherein the oocyte has been removed. The aged oocyte is combined with the young ovarian follicle that does not comprise an oocyte to generate the chimeric ovarian follicle.

In one example, the donor somatic cells comprise granulosa cells.

In another aspect, the present invention refers to a method of treating infertility or improving fertility in a female subject in need thereof, comprising:
a) obtaining an ovarian follicle from the subject and isolating an aged oocyte from an ovarian follicle:
b) obtaining an ovarian follicle from a donor and removing an oocyte from the donor ovarian follicle:
c) transplanting the aged oocyte from a) with the ovarian follicle without the oocyte from b) to generate a chimeric ovarian follicle:
d) culturing the chimeric ovarian follicle to generate a mature chimeric ovarian follicle; and
e) i) implanting the chimeric ovarian follicle into the female subject to treat infertility or improve fertility: or
ii) ovulating a mature oocyte from the mature chimeric ovarian follicle and isolating the mature oocyte for in vitro fertilization.

In one example, the female subject may be infertile, less infertile, or may suffer from conditions associated with fertility. The conditions associated with fertility may comprise but are not limited to early menopause and oocyte maturation abnormalities.

The mature chimeric ovarian follicle may be implanted into the female subject via means that are known in the art. In some examples, the quality of the mature chimeric ovarian follicle may be determined prior to implantation in the female subject.

In one example, the mature oocyte may be ovulated by contacting the mature chimeric ovarian follicle with hCG. The mature oocyte may be isolated via means that are known in the art and may subsequently be used for in vitro fertilization. In one example, the oocyte is isolated manually or through enzymatic digestion.

In one example, the female subject is a female mammalian subject. In one example, the mammalian subject is a human or rodent.

In one aspect, provided herein is a use of a chimeric ovarian follicle in the manufacture of a medicament for treating infertility or improving fertility in a female subject in need thereof, wherein the chimeric ovarian follicle comprises an oocyte from the female subject and donor somatic cells.

A chimeric ovarian follicle comprising an oocyte from a female subject and donor somatic cells for use in therapy.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### Experimental Section

Non-limiting examples of the invention and comparative examples will be further described in greater detail by reference to specific Examples, which should not be construed as in any way limiting the scope of the invention.

### Materials and Methods

### Isolation, Encapsulation and Culturing of Follicle

Ovaries were dissected from 2-3 month-old (young) or 14-18 month-old (aged) female mice. Individual follicles were obtained by breaking down the ovaries into small pieces with a 26 G needle and incubating them in Dissection Media containing L-15 (Thermo Scientific, #11415064) with 1% fetal bovine serum (FBS) and 100 U/ml penicillin/streptomycin supplemented with 2 mg/ml collagenase (SCR103, Sigma) and 10 U/ml DNAse (D4263, Sigma) for 30-40 minutes. Secondary follicles were then isolated and placed in Maintenance Media containing αMEM (Glutamax, Thermo Scientific #32561102) with 5% FBS and 100 U/ml penicillin-streptomycin for 2 hours before encapsulation. Each follicle was individually encapsulated in a 4.5 μl drop of Alginate-rBM IPN bead. Following a single wash with Maintenance Media, the IPN beads were placed in either 96-well or 24-well plates. Follicles were then cultured in Growth Media, composed of a 1:1 mixture of αMEM Glutamax and F-12 Glutamax, enriched with 5% FBS, 100 mIU/ml follicle-stimulating hormone (FSH, Sigma), 5 μg/ml insulin, 5 μg/ml transferrin, and 5 μg/ml selenium. The follicle culture was maintained at 37° C., with half of the growth media refreshed every other day.

### Generation of Reconstituted Chimeric Follicle (RCF)

To prepare oocytes for transplantation, grown oocytes were denuded from secondary follicles using either 0.25% trypsin treatment or repeated mouth pipetting with a fine oocyte-sized glass pipette. Denuded oocytes were placed in Maintenance Media containing αMEM (Glutamax, Thermo Scientific #32561102) with 5% FBS and 100 U/ml penicillin-streptomycin until use.

To generate RCFs, a 27 G needle tip was bent by gently dragging it across a petri dish surface. Firstly, this bent needle tip was used to anchor and stabilize the r-follicles designated for transplantation, preventing unwanted movement during the RCFs formation process. Subsequently, the follicle was gently pierced with a fine oocyte-sized glass mouth pipette and the oocyte was carefully aspirated, leaving an empty oocyte pocket within the follicle. A denuded oocyte using the same fine oocyte-sized glass mouth pipette was promptly picked up and gently released into the follicle pocket. The same process was used for the next follicle. After generating 3-10 RCFs, these RCFs were immediately encapsulated in alginate-reconstituted basement membrane (rBM) interpenetrating network (Alg-rBM IPN) beads and cultured in Growth Media supplemented with 100 ng/ml GDF9. On the following day, half of the media was replaced with fresh Growth Media without GDF9.

### Oocyte Maturation, Fertilization, and Embryo Culture

Follicles were extracted from the Alg-rBM IPN beads using 10 IU/mL of alginate lyase (A1603, Sigma). The oocytes that were cultured within the follicles were denuded and matured in Maturation Medium (αMEM with 10% FBS, 1.5 IU/ml human chorionic gonadotropin [hCG], 10 ng/ml epidermal growth factor [EGF], and 10 mIU/ml FSH) for 16 hours at 37° C. in 5% CO2 in air.

Oocytes matured in vitro were used for in vitro fertilization (IVF). To prepare for IVF, caudae epididymides from two 3-4 month old ICR male mice were lanced in 2 drops (100 μL/drop) of FERTIUPR Preincubation medium (KYD-002-05-EX, Cosmo Bio) under mineral oil to release sperm, followed by capacitation for 1 hour at 37° C. and 5% CO₂. MII oocytes were then placed in 100 μL of mHTF medium (KYD-008-02-EX-X5, Cosmo Bio) for 30 minutes at 37° C. and 5% CO₂ before being fertilized with 3 μL of sperm suspension. After 6 hours of fertilization, wash the zygotes 3 times with mHTF and culture them in 50 μl mHTF overnight. Next, transfer the resulting 2-cell stage embryos to Continuous Single Culture Media (CSCM-C, Fujifilm #90165) and culture them for 4 days at 37° C., or transfer them to pseudo-pregnant female mice.

### Embryo Transfer

Female B6C3HF2 mice (3-6 months old) were employed as surrogate mothers in this study. Recipient female (0.5 dpc Pseudo pregnant mouse) was anesthetized with 2.5% Avertin. Animal was checked for loss of pedal reflex and sprayed down with ethanol. A small incision is then made along the dorsal midline of the skin and muscle layer of the left side of the animal. A drop of Epinephrine was placed on the muscle layer prior to incision to prevent excessive bleeding. The ovarian fat pad was seized with forceps and pulled through the incision, carrying with it the ovary, the oviduct, and the upper part of the left uterine horn. Under a stereomicroscope with optic fiber light, a tear is made at the ovarian bursa to expose the infundibulum of oviduct and prepare for oviduct transfer. Tip of Transfer pipette (loaded with HEPES-buffered media and embryos flanked by “Air bubbles”) is inserted into the infundibulum and embryos are blown into the oviduct with a mouth pipette. Air bubbles within the ampulla indicate successful transfer. Uterus, oviduct, and ovary are replaced back inside the body cavity. The muscle and skin layer are sutured. Earlier steps are repeated to transfer additional embryos to the right oviduct. A total of 18 to 20 two-cell stage embryos were transferred into one recipient female.

### Immunofluorescence

Oocytes, follicles, or embryos were fixed in 4% paraformaldehyde in phosphate-buffered saline (PBS) for 40 minutes and then incubated in a membrane permeabilization solution (0.5% Triton X-100) for 40 minutes. After overnight blocking with 10% bovine serum albumin in PBS, the samples were incubated with the primary antibody overnight at 4° C., followed by incubation with a secondary antibody at room temperature for 1-3 hours.

### Confocal Microscopy

For live follicles, images were acquired with a Zeiss LSM710 confocal microscope with 10× or 20× objectives. For live oocytes, images were acquired with Zeiss LSM980 microscope using a 40× C-Apochromat 1.2-numerical aperture water-immersion objective. Follicles and oocytes were maintained at 37° C. with 5% CO2 during imaging. For fixed follicles or oocytes, images were acquired using the Zeiss LSM980 microscopes and processed after acquisition using ZEN (Zeiss).

### Results

### Example 1

### Development of Ovarian Follicle

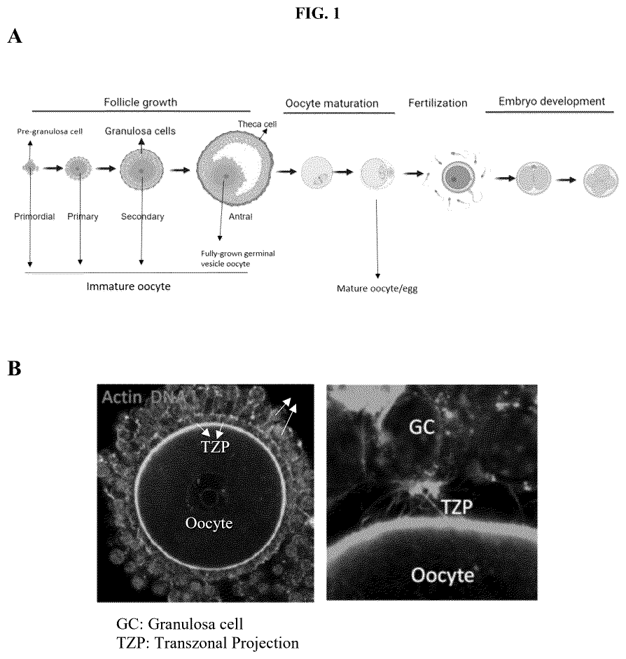

The ovarian follicle develops from the primordial stage to the primary stage, the secondary stage, and the antral stage during folliculogenesis. Somatic cells comprising pre-granulosa cells, granulosa cells and theca cells promote the development of the oocyte within the ovarian follicle and the ovarian follicle grows and matures at antral stage. The ovarian follicle at the antral stage comprises a fully-grown germinal vesicle oocyte which matures upon meiotic maturation. The mature oocyte is subsequently released from the ovarian follicle for fertilization (FIG. 1A). The somatic cells of the ovarian follicle comprise granulosa cells, cumulus cells and theca cells, and the ovarian follicle grows and develops with the oocyte. These somatic cells nourish the oocyte throughout development through specialized structures named Transzonal Projections (TZPs) (FIG. 1B).

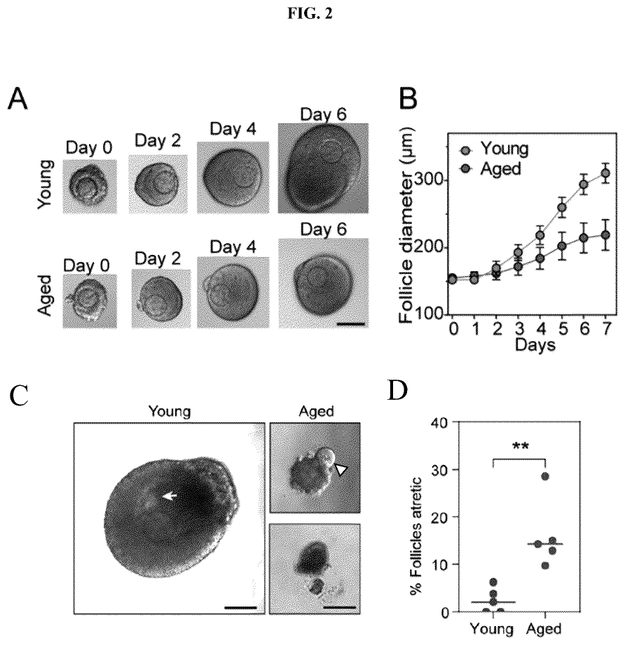

The effect of aging on the quality of the ovarian follicles was investigated. The follicles were cultured in a three-dimensional culture which allows the quantification of the quality attributes between the young and aged follicles, which are the size and death rate of the follicles obtained from young and aged mice. The young follicles grew at a slower rate and were larger in diameter as compared to the aged follicles 6 days after culturing in the three-dimensional culture (FIGS. 2A and 2B). The young follicles maintained the three-dimensional structure with proliferation of GCs and antrum formation while the aged follicles were more prone to undergo atresia during in vitro culture as compared to young follicles. It was observed that there was premature release of the oocytes from the atretic follicles due to the disruption of the contact between the oocyte and the granulosa cells, or apoptotic or dead oocytes in the atretic follicles (FIG. 2C). Further, the death rate of the aged follicles in the three-dimensional culture was significantly higher as compared to the young follicles (FIG. 2D).

Collectively, this indicates that young follicles are of higher quality than the aged follicles.

### Example 2

### Creation of Reconstituted Chimeric Follicles

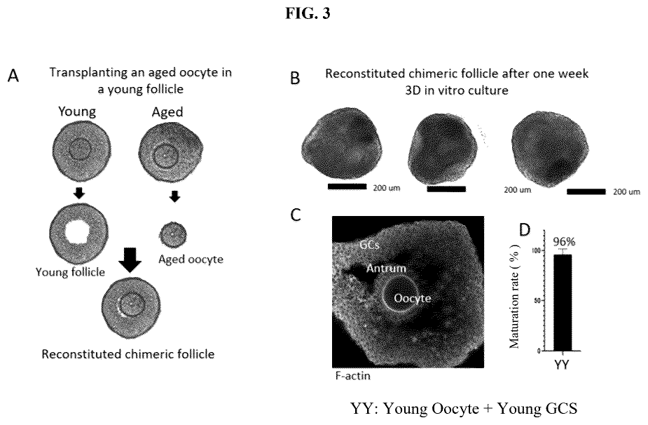

Reconstituted chimeric ovarian follicles were generated to rejuvenate aged oocytes through the implantation of the aged oocyte into a young follicle that does not comprise an oocyte. (FIG. 3A).

The results demonstrated high quality reconstituted chimeric follicles displaying actively dividing somatic cells and well formed TZPs and ZPs, and these reconstituted chimeric follicle were able to progress to the antral stage. These reconstituted chimeric follicles, which comprise young granulosa cells and aged oocytes grew to their maximum potential size of 300 μm in diameter following a one-week three-dimensional culture, which is indicative of a high-quality ovarian follicle at the pre-ovulatory state (FIG. 3B). Live cell image showed that the reconstituted chimeric follicle comprising an aged oocyte and young granulosa cells developed an antrum and maintained actively dividing cells, and developed TZPs (FIG. 3C). The number of mature oocytes (MII oocytes) was calculated and the result showed that about 96% of mature oocytes were released from reconstituted chimeric ovarian follicles comprising young oocytes and young somatic cells (YY) during ovulation (FIG. 3D).

### Example 3

### Rejuvenation of Aged Oocytes

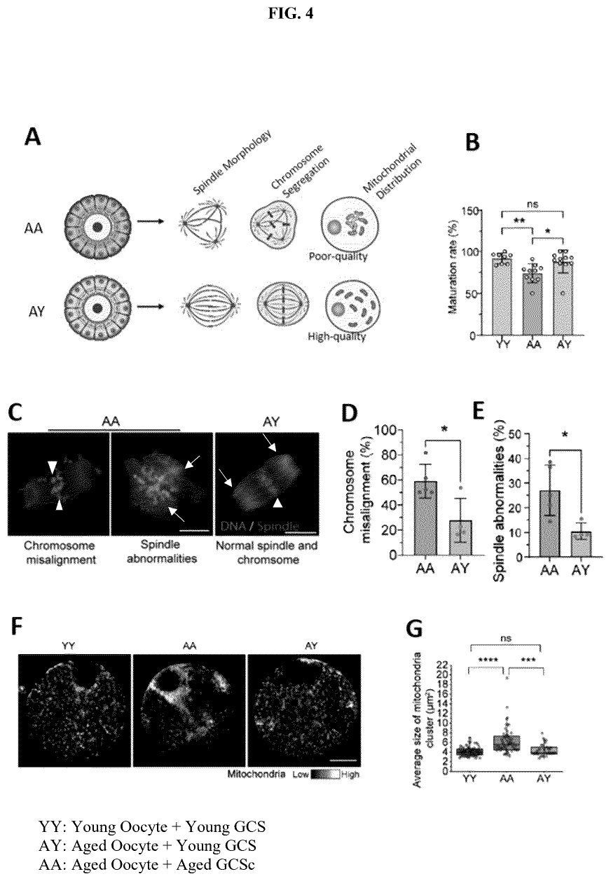

The chimeric follicle technique improved the quality of aged oocytes as gauged by the three criteria depicted in FIG. 4A which comprise spindle morphology, chromosome segregation and mitochondrial distribution. In addition, the maturation rate of the oocytes was also calculated. The maturation rate of the oocytes was significantly increased in the reconstituted chimeric follicle comprising an aged oocyte and young granulosa cells (AY) as compared to the chimeric follicle comprising an aged oocyte and aged granulosa cells (AA) (FIG. 4B). The quality of the aged oocyte in AY reconstituted chimeric follicle was significantly higher as compared to the aged oocyte in the AA reconstituted chimeric follicle (FIG. 4C). The aged oocyte in the AA reconstituted chimeric follicle displayed chromosome misalignment and spindle abnormalities while the reconstituted AY chimeric follicle displayed normal spindle morphology and normal chromosome alignment.

The percentages of the rate of spindle abnormalities and misalignment of chromosome were calculated. The percentage of chromosome misalignment (FIG. 4D) and spindle abnormalities (FIG. 4E) in the oocytes of the AY reconstituted chimeric follicles was significantly reduced as compared to the oocytes of the AA reconstituted chimeric follicles. Mitochondrial distribution, was improved in AY reconstituted chimeric follicles when compared to either YY reconstituted chimeric follicles or AA reconstituted chimeric follicles (FIG. 4F). The average size of mitochondria cluster in oocytes from reconstituted chimeric follicles was calculated and the size of mitochondria cluster in oocytes from AY reconstituted chimeric follicle was significantly reduced as compared to the oocytes from AA reconstituted chimeric follicle (FIG. 4G). These findings indicated that the quality of the aged oocytes in the AY reconstituted chimeric follicle was significantly higher as compared to aged oocytes in the AA reconstituted chimeric follicle and young granulosa cells improved the quality of aged oocytes.

### Example 4

### Effect of Young Granulosa Cells on Aneuploidy in Aged Oocytes

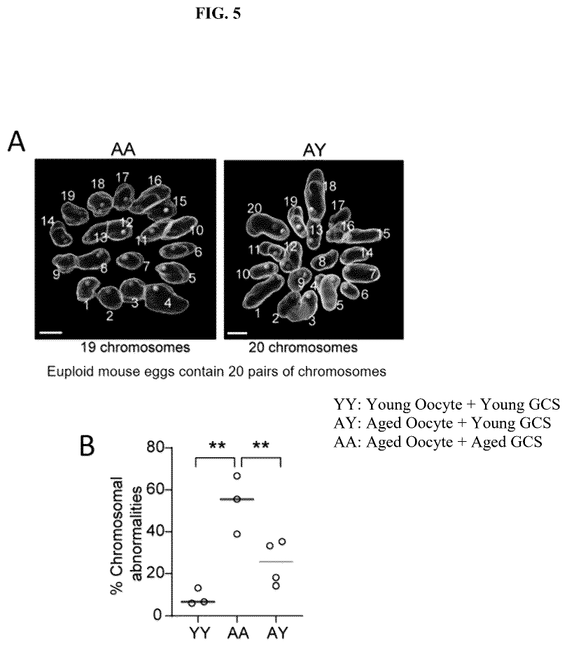

One main cause of increased risk for miscarriage in aged female subjects is the increased rates of aneuploidy in their eggs. The effect of using young granulosa cells to rejuvenate aged oocytes on aneuploidy was investigated in mice. Euploid mouse eggs contain pairs of chromosomes and therefore have 40 centromeres. Immunofluorescence analysis showed that the aged oocytes in the AY reconstituted chimeric follicles contained 20 pairs of chromosomes and 40 centromeres while the oocytes in the AA reconstituted chimeric follicles contained abnormal number of chromosomes and centromeres (FIG. 5A). The percentage of the chromosomal abnormalities was calculated, and the results showed that young GCs decreased the percentage of chromosomal abnormalities in aged oocytes as compared to aged oocytes grown with aged GCs (FIG. 5B).

### Example 5

### Effect of Young Granulosa Cells on Embryo Development

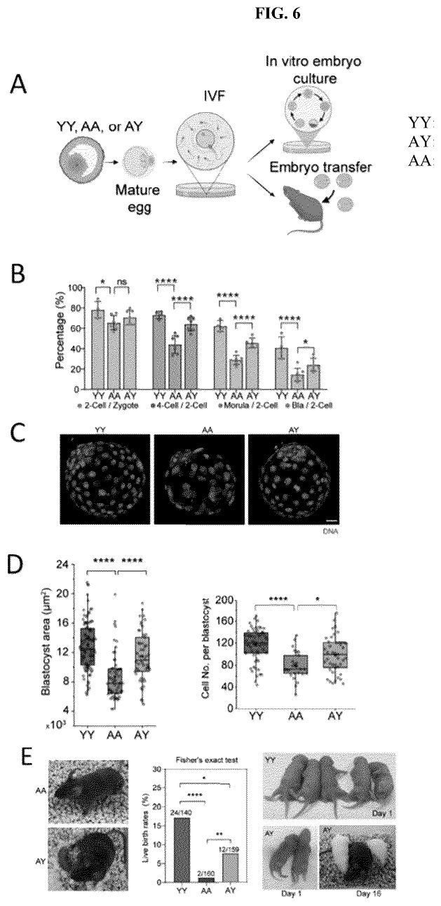

As the young GCs reduced aneuploidy in aged oocytes, the effect of the young follicular somatic environment on embryo development was studied. To assess if the young follicular somatic environment facilitates the development of the embryo, in vitro fertilization was performed and the early embryonic developmental stages of the fertilized eggs were monitored (FIG. 6A). The results demonstrated that aged oocytes grown with young GCs in AY reconstituted chimeric follicles had dramatically higher percentage of blastocyst formation rates compared to aged oocytes growth with aged GCs in AA reconstituted chimeric follicles (FIG. 6B). In addition, the percentage of blastocyte formation in aged oocytes in AY reconstituted chimeric follicles was significantly higher as compared to the aged oocyte in AA reconstituted chimeric follicles (FIGS. 6C and 6D). Subsequently, the live birth rate of the pups generated from the YY, AY and AA reconstituted chimeric follicles was calculated. The results showed that the live birth rate of the pups generated from the AY reconstituted chimeric follicle was significantly increased as compared to that generated from the AA reconstituted chimeric follicle (FIG. 6E).

### Example 6

Mitochondria Transport from Somatic Cells to Oocyte

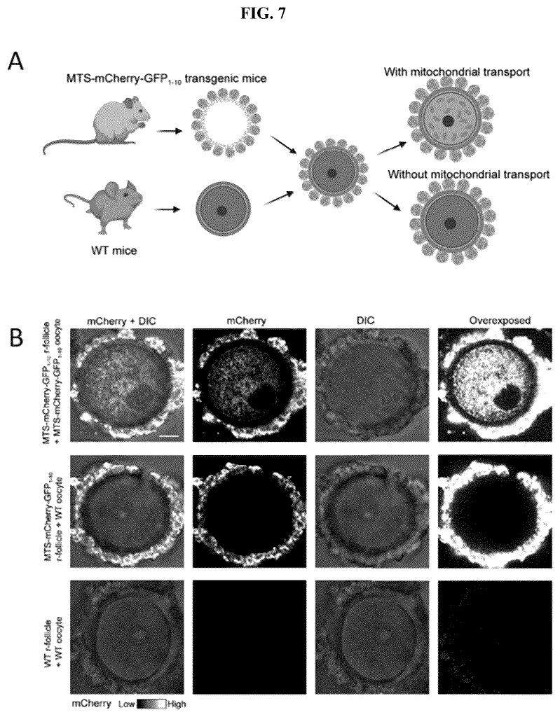

The existing method to rejuvenate aged oocytes through mitochondrial transfer by replacing the damaged mitochondria in the eggs with healthy mitochondria from another egg of a female donor is controversial as the developing embryo contains a third unknown donor mitochondrial DNA and poses safety concerns. Therefore, the mitochondria (mtDNA) transport from the surrounding somatic cells to the oocyte was investigated. The MTS-mCherry transgenic mice expressing mitochondria-targeted mCherry was utilized to allow the unambiguous identification of the origins of the mitochondria. The wild-type oocytes were implanted into follicles that were isolated from MTS-mCherry transgenic mice (FIG. 7A). After 5 days of culture, the oocytes were isolated from the reconstituted chimeric follicle and the oocyte tissues were stained with anti-mCherry antibodies to determine whether the mCherry-positive mitochondria were present. The results clearly showed that no mCherry-positive mitochondria were present in the wild-type oocytes after co-culturing with MTS-mCherry somatic cells (FIG. 7B). The aged oocyte grown with young granulosa cells did not contain DNA from another female subject, thereby confirming that mitochondrial DNA was not transferred to the developing embryo.

### EQUIVALENTS

The foregoing examples are presented for the purpose of illustrating the invention and should not be construed as imposing any limitation on the scope of the invention. It will readily be apparent that numerous modifications and alterations may be made to the specific embodiments of the invention described above and illustrated in the examples without departing from the principles underlying the invention. All such modifications and alterations are intended to be embraced by this application.

What is claimed is:

1. An in vitro method of rejuvenating an aged oocyte comprising combining the aged oocyte with a donor ovarian follicle that does not comprise an oocyte to generate a chimeric ovarian follicle.

2. The in vitro method according to claim 1, wherein the aged oocyte is obtained from an aged ovarian follicle comprising somatic cells that are less capable or not capable of promoting development of the oocyte.

3. The in vitro method according to claim 2, wherein the somatic cells that are less capable or not capable of promoting development of the oocyte are characterized by one or more phenotypes selected from the group consisting of higher reactive oxidative stress level, lower mitochondria membrane potential, increased DNA damage and apoptosis, decreased ATP levels, absence of or decreased cohesion, or a combination thereof, relative to somatic cells that are capable of promoting development of the oocyte.

4. The in vitro method according to any one of claims 1-3, wherein the donor ovarian follicle is a young follicle.

5. The in vitro method according to any one of claims 1-4, wherein the donor ovarian follicle comprises somatic cells that are capable of promoting development of the oocyte.

6. The in vitro method according to claim 5, wherein the somatic cells that are capable of promoting development of the oocyte have one or more predetermined phenotypes selected from the group consisting of lower reactive oxidative stress level, higher mitochondria membrane potential, lesser DNA damage and apoptosis, increased ATP levels, increased cohesion, or a combination thereof, relative to somatic cells that are less capable or not capable of promoting development of the oocyte.

7. The in vitro method according to any one of claims 1-6, further comprising culturing the chimeric ovarian follicle to generate a mature chimeric ovarian follicle and inducing ovulation of a mature oocyte from a mature chimeric ovarian follicle.

8. The in vitro method according to claim 7, wherein the mature chimeric ovarian follicle is an antral stage chimeric follicle.

9. The in vitro method according to claim 7 or 8, wherein the mature chimeric ovarian follicle comprises a fully grown germinal vesicle (GV) stage oocyte and somatic cells.

10. The in vitro method according to any one of claims 3-9, wherein the somatic cells comprise granulosa cells.

11. The in vitro method according to any one of claims 1-10, wherein the aged oocyte is a mammalian oocyte and the donor ovarian follicle is a mammalian ovarian follicle.

12. The in vitro method according to claim 11, wherein the mammalian oocyte is a human or rodent oocyte, and wherein the mammalian donor ovarian follicle is a human or rodent ovarian follicle.

13. A chimeric ovarian follicle, comprising an aged oocyte and donor somatic cells.

14. The chimeric ovarian follicle according to claim 13, wherein the aged oocyte is obtained from an aged ovarian follicle comprising somatic cells that are less capable or not capable of promoting development of the oocyte.

15. The chimeric ovarian follicle according to claim 13 or 14, wherein the donor somatic cells are obtained from an ovarian follicle comprising somatic cells that are capable of promoting development of the oocyte.

16. The chimeric ovarian follicle according to claim 15, wherein the donor ovarian follicle is a young follicle.

17. The chimeric ovarian follicle according to any one of claims 13-16, wherein the somatic cells are granulosa cells.

18. A method of treating infertility or improving fertility in a female subject in need thereof, comprising:
a) obtaining an ovarian follicle from the female subject and isolating an aged oocyte from an ovarian follicle;
b) obtaining an ovarian follicle from a donor and removing an oocyte from the donor ovarian follicle;
c) transplanting the aged oocyte from a) with the ovarian follicle without the oocyte from b) to generate a chimeric ovarian follicle;
d) culturing the chimeric ovarian follicle to generate a mature chimeric ovarian follicle; and
e) i) implanting the chimeric ovarian follicle into the female subject to treat infertility or improve fertility; or
ii) ovulating a mature oocyte from the mature chimeric ovarian follicle and isolating the mature oocyte for in vitro fertilization.

19. The method according to claim 18, wherein the female subject is a female mammalian subject.

20. The method according to claim 19, wherein the mammalian subject is a human or a rodent.
